(19) United States
(12) Patent Application Publication
(10) Pub. No.: US 2010/0280506 A1
(43) Pub. Date: Nov. 4, 2010

(54) METHOD AND APPARATUS FOR MEDICAL TREATMENT UTILIZING LASER IRRADIATION

(76) Inventors: Valerii Kanevskiy, Kiev (UA); Volodymyr Solodovnikoff, Kiev (UA); Volodymyr Kapustin, Kiev (UA)

Correspondence Address: MAIER & MAIER, PLLC, 1000 DUKE STREET, ALEXANDRIA, VA, 22314, US

(21) Appl. No.: 12/434,004

(22) Filed: May 1, 2009

(51) Int. Cl. A61B 18/22; H01S 3/30

(52) U.S. Cl. 606/15; 372/5

(57) ABSTRACT

According to at least one exemplary embodiment a method and apparatus for medical treatment utilizing laser irradiation is disclosed. The method may comprise generating an electromagnetic radiation pulse by a laser, directing the radiation to an area of the human body where treatment is desired; applying the radiation to the area of the human body; and inducing conformational changes in organic molecules having metal ions in their structure.

### BACKGROUND

Conventional medical methods of treating ailments such as cancerous tumors, autoimmune diseases, and infectious pathologies have included chemotherapy, radiation therapy, invasive surgery, and pharmaceutical drugs. Many of these treatments may have negative physical and emotional effects on the patient, as well as potentially dangerous side effects. For example, chemotherapeutical methods of treatment are toxic to any rapidly-dividing cells, such as hair follicles, and may also induce fatigue and nausea in the patient, while invasive surgery carries the potential risks of infection, bleeding, and other life-threatening consequences.

Recent advancements in medicine have demonstrated that laser technologies may have applications in medical treatment, for example, in the oncological, dermatological and opthalmological fields. However, a safe and non-toxic method of treating cancer, autoimmune diseases, and infectious pathologies is needed.

### SUMMARY

According to at least one exemplary embodiment, a method for medical treatment utilizing laser irradiation is disclosed. The method may comprise using generating an electromagnetic pulse by a laser; directing the radiation to an area of the human body where treatment is desired; applying the radiation to the human body; and inducing conformational changes in organic molecules which have metal ions in their structure.

In another exemplary embodiment, an apparatus for medical treatment is disclosed. The apparatus may comprise a laser emitting an electromagnetic radiation pulse and an optical fiber coupled to the laser for directing the radiation to an area of the human body where treatment is desired.

### BRIEF DESCRIPTION OF THE FIGURES

Advantages of embodiments of the present invention will be apparent from the following detailed description of the exemplary embodiments thereof, which description should be considered in conjunction with the accompanying drawings in which:

FIG. 1 is a flowchart of a method of medical treatment utilizing laser irradiation according to at least one exemplary embodiment.

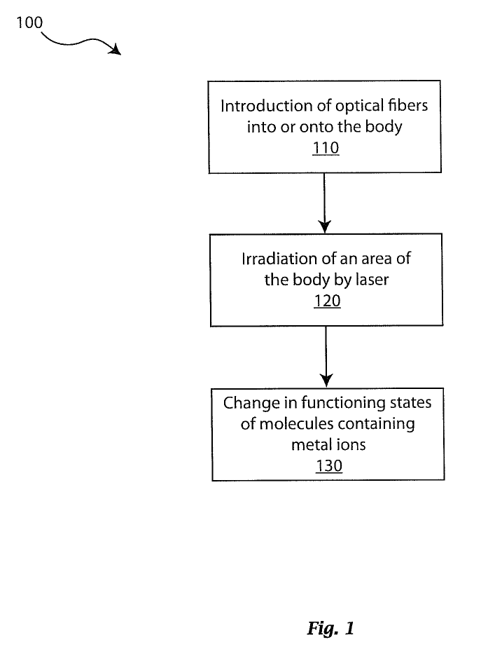

### DETAILED DESCRIPTION

Aspects of the present invention are disclosed in the following description and related figures directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

In one exemplary embodiment, a method for treating medical conditions, such as, for example, autoimmune diseases, infectious diseases and cancer using laser irradiation is disclosed. The method may utilize a laser capable of generating a pulse having a duration in the range of about 75 to 125 femtoseconds, such as, for example about 100 femtoseconds, and having an impulse frequency in the range of about 50 to 100 megahertz, such as, for example, about 76 megahertz. The laser may be capable of generating radiation with a power density in the range of about 10 milliwatts per square centimeter to 70 milliwatts per square centimeter. The laser may be a highly tunable laser or a laser capable of generating radiation having wavelengths in the range of about 370 to 390 nanometers, such as, for example, about 381.5 nanometers to 385.5 nanometers. In one embodiment, the laser may be a highly tunable, solid state laser, such as, for example, a titanium-doped sapphire laser. The spectral characteristics of the laser may further be varied such that the parameters of the electromagnetic radiation pulse generated by the laser best correspond to the medical condition to be treated.

Radiation emitted by the laser may be applied to an area of the human body where treatment is desired. Such areas may include, for example, the frontal projection surface area of the thymus, in the area of the front part of the chest of the human body, or, for example, an area having approximately a 5-centimeter radius around the upper center part of the sternum. Radiation may also be applied to internal areas of the human body where treatment is desired, such as, for example, the surface of the thymus, the surface of a tumor, the nervus vagus area, or the innervation of the liver. Radiation may be applied to the surface of the thymus during a surgical procedure such as, for example, a thoracotomy procedure, or for example, by introducing a subclavian catheter into the large saphenic vein, proximate to the thymus.

In one exemplary embodiment, optical fibers may be used to direct the radiation emitted by the laser to a desired part of the body. Optical fibers may be introduced into the body via surgical or non-surgical procedures, such that the proximal end of the optical fiber is coupled to the laser and the distal end of the optical fiber is located proximate to the surface of the desired organ or desired tissue. The distal end of the optical fiber may also be located on the external surface of the epidermis, or underneath and proximate to the dermis. Optical fibers may also be introduced into the body via injection or syringe needles.

The application of radiation to desired parts of the body via the described method may have the result of inducing changes in organic molecules having metal ions in their structure. Specifically, the application of radiation to desired parts of the body via the described method may have the result of inducing changes in the functioning states of enzymes, such as, for example, enzymes containing metal ion ligands. Such enzymes may include, for example, principal thymal enzymes containing single or multiple metal ion ligands in their active sites. The metal ion ligands may include, for example, zinc, iron, calcium or magnesium ions. Such enzymes may include, for example, IL-2 inducible T-cell kinase and resting lymphocyte kinase. The application of radiation to desired parts of the body via the described method may also have the result of inducing changes in the functioning states of hormones, such as, for example, hormones containing single and multiple metal ions in their structures, such as, for example, zinc ions. Such hormones may include, for example, zinc-binding thymulin.

The radiation emitted by the laser may excite electrons in the metal ion ligand to higher orbital states, which may induce a conformational geometric change in the three-dimensional structure of the enzyme and may consequently alter the activity of the enzyme. Change in the activity of the enzyme may then trigger various protein signaling pathways, resulting in therapeutic effects against cancer, autoimmune and infectious diseases.

The effects of the treatment method described herein may include, for example, changes in the concentration and activity levels of immune cells in the blood, such as T-Helper cells, T-cytotoxic cells, T-regulatory cells and NK cells. Such changes in activity levels and concentrations may last for time periods of about 10 to 14 days. Effects may also include the normalization of concentration and activity level of T-Helper cells, T-cytotoxic cells, T-regulatory cells and NK cells when abnormalities in concentration and activity levels are present. Effects may also include normalization of general state of health and normalization of biochemical liver parameters in patients with HBV, HCV, HIV/AIDS and neuroinfections, as well as an increase in the number of T-helper cells in patients with HIV/AIDS. Effects may also include an increase in the binding of zinc to thymulin molecules and activation of calcioneurin, triggering an immune response signaling pathway. Oncological effects may include, for example, inhibition of activity of matrix metal proteinases such as MMP-2 and MMP-9, induction of tumor cell apoptosis, and inhibition of metastase proliferation. Immunotherapeutic effects may also include, for example, an increase in the quantity of NK cells and a decrease in the quantity of T-regulatory cells, such as for example, cells having clusters of differentiation CD4+ and CD25+.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A method for medical treatment utilizing laser irradiation, comprising:
generating an electromagnetic radiation pulse by a laser;
directing the radiation to an area of the human body where treatment is desired;
applying the radiation to the area of the human body; and
inducing conformational changes in organic molecules having metal ions in their structure.

2. The method of claim 1, further comprising directing the radiation through an optical fiber to an area of the human body where treatment is desired.

3. The method of claim 1, further comprising applying the radiation to the thymus.

4. The method of claim 1, further comprising applying the radiation to the nervus vagus.

5. The method of claim 1, further comprising applying the radiation to the innervation of the liver.

6. The method of claim 1, wherein the electromagnetic radiation pulse has a duration of about 75 femtoseconds to about 125 femtoseconds.

7. The method of claim 6, wherein the electromagnetic radiation pulse has a duration of about 100 femtoseconds.

9. The method of claim 1, wherein the electromagnetic radiation pulse has an impulse frequency of about 50 mHz to about 125 mHz.

10. The method of claim 9, wherein the electromagnetic radiation pulse has an impulse frequency of about 76 mHz.

11. The method of claim 1, wherein the electromagnetic radiation pulse has a power density of about 10 mW/cm² to about 70 mW/cm².

12. The method of claim 1, wherein the electromagnetic radiation pulse has a wavelength of about 370 nm to about 390 nm.

13. The method of claim 12, wherein the electromagnetic radiation pulse has a wavelength of about 381.5 nm to about 385.5 nm.

14. The method of claim 1, wherein the organic molecules are enzymes containing metal ion ligands.

15. The method of claim 1, wherein the organic molecules are hormones containing metal ions in their structure.

16. The method of claim 1, wherein the laser is a highly tunable solid state laser.

17. The method of claim 16, wherein the laser is a titanium-doped sapphire laser.

18. An apparatus for medical treatment, comprising:
a titanium-doped sapphire laser, said laser emitting an electromagnetic radiation pulse having a duration of about 75 femtoseconds to about 125 femtoseconds, an impulse frequency of about 50 mHz to about 100 mHz, a power density of about 10 mW/cm² to about 70 mW/cm², and a wavelength in the range of about 370 nm to about 390 nm; and
an optical fiber for directing the electromagnetic radiation pulse, said having a first end coupled to the laser and a second end applied to an area of the human body where treatment is desired.

19. The apparatus of claim 18, wherein the electromagnetic radiation pulse has a duration of about 100 femtoseconds.

20. The apparatus of claim 18, wherein the electromagnetic radiation pulse has an impulse frequency of about 76 mHz.

21. The apparatus of claim 18, wherein the electromagnetic radiation pulse has a wavelength of about 381.5 nm to about 385.5 nm.

22. An apparatus for treating medical conditions by inducing conformational changes in organic molecules having metal ions in their structure, comprising:
means for emitting an electromagnetic radiation pulse;
means for directing the radiation to an area of the human body where treatment is desired; and
means for applying the radiation to the area of the human body.
